(19) United States
(12) Patent Application Publication
(10) Pub. No.: US 2024/0012891 A1
(43) Pub. Date: Jan. 11, 2024

(54) INFORMATION ACQUISITION SYSTEM, INFORMATION ACQUISITION METHOD, AND STORAGE MEDIUM

(71) Applicant: NEC CORPORATION, Tokyo (JP)

(72) Inventor: Masanori MIZOGUCHI, Tokyo (JP)

(73) Assignee: NEC CORPORATION, Tokyo (JP)

(21) Appl. No.: 18/371,216

(22) Filed: Sep. 21, 2023

Related U.S. Application Data
(63) Continuation of application No. 16/636,397, filed on Feb. 4, 2020.

(30) Foreign Application Priority Data
Aug. 10, 2017 (JP) 2017-155190

(51) Int. Cl. G06F 21/32; G06Q 20/40; G06V 40/19; G06V 40/18; G07C 9/25

(52) U.S. Cl. CPC G06F 21/32; G06Q 20/40145; G06V 40/19; G06V 40/197; G07C 9/25

(57) ABSTRACT

An example embodiment includes an acquisition unit that acquires a first image used for biometrics authentication of an authentication target and a second image that is based on a light projected onto and reflected by a body of the authentication target and includes information used for a different purpose from the biometrics authentication of the authentication target.

This application is a Continuation of U.S. application Ser. No. 16/636,397, filed on Feb. 4, 2020, was is a National Stage of International Application No. PCT/JP2018/029684 filed Aug. 7, 2018, claiming priority based on Japanese Patent Application No. 2017-155190 filed Aug. 10, 2017, and the contents each of which are herein incorporated by reference in their entireties.

### TECHNICAL FIELD

The present invention relates to an information acquisition system, an information acquisition method, and a storage medium.

### BACKGROUND ART

Patent Literature 1 discloses a monitoring system including a biometrics authentication device that reads biometrics information on a user and a wireless terminal location information acquisition device that acquires location information on a wireless terminal. The monitoring system determines whether or not to permit the user to enter a room based on the biometrics information and the location information.

### CITATION LIST

### Patent Literature

PTL 1: Japanese Patent Application Laid-Open No. 2016-206904

### SUMMARY OF INVENTION

### Technical Problem

In the monitoring system of Patent Literature 1, both of the biometrics authentication device and the wireless terminal location information acquisition device are devices that acquire information used for biometrics authentication or assistance to biometrics authentication. In the configuration of Patent Literature 1, to further acquire information used for a different purpose from biometrics information, another information acquisition unit is required.

The present invention has been made in view of the problem described above and intends to provide an information acquisition system, an information acquisition method, and a storage medium that can acquire information used for a different purpose from biometrics authentication in addition to acquisition of information used for biometrics authentication.

### Solution to Problem

According to one example aspect of the present invention, provided is an information acquisition system including an acquisition unit that acquires a first image used for biometrics authentication of an authentication target and a second image that is based on a light projected onto and reflected by a body of the authentication target and includes information used for a different purpose from the biometrics authentication of the authentication target.

According to another example aspect of the present invention, provided is an information acquisition method including: acquiring a first image used for biometrics authentication of an authentication target; and acquiring a second image that is based on a light projected onto and reflected by a body of the authentication target and includes information used for a different purpose from the biometrics authentication of the authentication target.

According to yet another example aspect of the present invention, provided is a storage medium storing a program that causes a computer to perform: acquiring a first image used for biometrics authentication of an authentication target; and acquiring a second image that is based on a light projected onto and reflected by a body of the authentication target and includes information used for a different purpose from the biometrics authentication of the authentication target.

### Advantageous Effects of Invention

According to the present invention, it is possible to provide an information acquisition system, an information acquisition method, and a storage medium that can acquire information used for a different purpose from biometrics authentication in addition to acquisition of information used for biometrics authentication.

### BRIEF DESCRIPTION OF DRAWINGS

FIG. 1 is a block diagram illustrating a general configuration of a first example embodiment.

FIG. 2 is a block diagram illustrating a hardware configuration example of a user terminal according to the first example embodiment.

FIG. 3 is a function block diagram of a payment server, a POS terminal, and a user terminal according to the first example embodiment.

FIG. 4 is a sequence diagram illustrating a payment process according to the first example embodiment.

FIG. 5 is a diagram schematically illustrating an iris image.

FIG. 6 is a diagram schematically illustrating a display unit on which a two-dimensional code is displayed.

FIG. 7 is a diagram schematically illustrating an image in which a two-dimensional code is reflected in an eye.

FIG. 8 is a block diagram illustrating a general configuration of a second example embodiment.

FIG. 9 is a function block diagram of an in-company system and a user terminal according to the second example embodiment.

FIG. 10 is a sequence diagram illustrating a user management process according to the second example embodiment.

FIG. 11 is a function block diagram of an entry/exit management system according to a third example embodiment.

FIG. 12 is a sequence diagram illustrating an entry/exit management process according to the third example embodiment.

FIG. 13 is a function block diagram of an information acquisition system according to a fourth example embodiment.

### DESCRIPTION OF EMBODIMENTS

Exemplary embodiments of the present invention will be described below with reference to the drawings. Throughout the drawings, the same or corresponding components are labeled with the same reference, and the description thereof may be omitted or simplified.

### [First Example Embodiment]

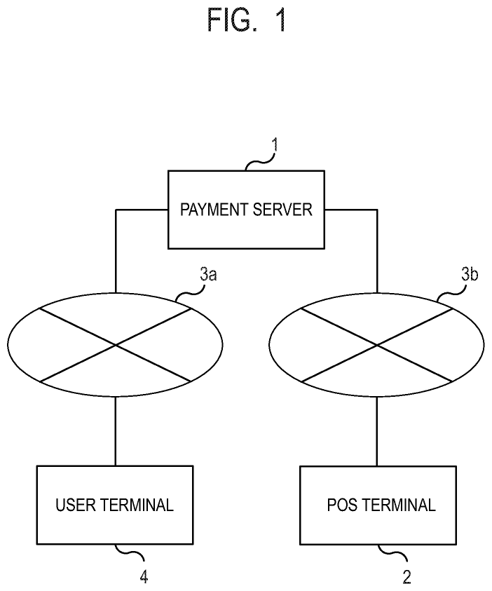

As a first example embodiment of the present invention, an example of a payment system that performs payment using iris authentication will be described. FIG. 1 is a schematic diagram illustrating a general configuration of the payment system according to the first example embodiment. The payment system includes a payment server 1, a Point Of Service (POS) terminal 2, and a user terminal 4. The payment system is a system that performs electronic payment when a user having the user terminal 4 purchases things at a shop where the POS terminal 2 is installed. The payment server 1 and the user terminal 4 are communicably connected via a network 3a, and the payment server 1 and the POS terminal 2 are communicably connected via a network 3b. The networks 3a and 3b each are an Internet Protocol (IP) network or the like. Each communication path via the networks 3a and 3b may be wired or wireless or may be the combination thereof.

The POS terminal 2 is a POS register that settles a check when an item is purchased at a shop, for example. The POS terminal 2 has a keypad used by a salesperson to input an item name or the like, a barcode scanner that reads a barcode of an item, a printer that prints a receipt, a display that displays a two-dimensional barcode or the like, or the like. An accounting process when an item is sold may be performed inside the POS terminal 2 or may be performed on a POS server (not illustrated) connected to communicate with the POS terminal 2. When an accounting process is performed on the POS server, the POS terminal 2 transmits information on the item to be sold to the POS server, and the POS server performs the accounting process.

The user terminal 4 is an information communication terminal such as a mobile phone, a smartphone, a tablet personal computer (PC), a laptop PC, or the like possessed by a shopping user. The user terminal 4 has a function of iris authentication that is a type of biometrics authentication. The user terminal 4 performs iris authentication by capturing an iris of the user who is an authentication target. The user terminal 4 has software that performs user authentication and claims payment for an item to be purchased in a shop to the payment server 1 via the network 3a. Note that, while the user terminal 4 and the POS terminal 2 may be connected for direct communication, the user terminal 4 and the POS terminal 2 may not be assumed to be connected for direct communication, as illustrated in FIG. 1.

The payment server 1 performs electronic payment with a credit card or the like for an item purchased by the user at a shop in response to a request by the user terminal 4. After completion of payment, the payment server 1 notifies the POS terminal 2 of the completion of payment via the network 3b.

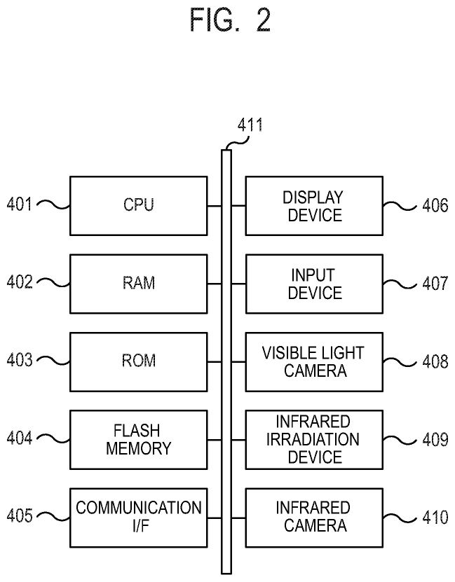

FIG. 2 is a block diagram illustrating a hardware configuration example of the user terminal 4 according to the first example embodiment. The user terminal 4 has a central processing unit (CPU) 401, a random access memory (RAM) 402, a read only memory (ROM) 403, and a flash memory 404 in order to implement a function as a computer that performs calculation and storage. Further, the user terminal 4 has a communication interface (I/F) 405, a display device 406, an input device 407, a visible light camera 408, an infrared irradiation device 409, and an infrared camera 410. The CPU 401, the RAM 402, the ROM 403, the flash memory 404, the communication I/F 405, the display device 406, the input device 407, the visible light camera 408, the infrared irradiation device 409, and the infrared camera 410 are connected to each other via a bus 411. Note that the display device 406, the input device 407, the visible light camera 408, the infrared irradiation device 409, and the infrared camera 410 may be connected to the bus 411 via drive devices (not illustrated) used for driving these devices.

While the components forming the user terminal 4 are depicted as an integral apparatus in FIG. 2, some functions of these components may be configured by an external device. For example, the visible light camera 408, the infrared irradiation device 409, or the infrared camera 410 may be an external device that is different from a portion that configures the function of the computer including the CPU 401 or the like.

The CPU 401 performs a predetermined operation in accordance with a program stored in the ROM 403, the flash memory 404, or the like and has a function of controlling respective components of the user terminal 4. The RAM 402 is formed of a volatile memory and provides a temporary memory area required for the operation of the CPU 401. The ROM 403 is formed of a nonvolatile memory and stores necessary information such as a program used for the operation of the user terminal 4. The flash memory 404 is formed of a nonvolatile memory, which is a storage device that stores an image captured by the visible light camera 408 and the infrared camera 410, an image of a matching target, feature data, or the like.

The communication I/F 405 is a communication interface based on a standard such as Wi-Fi (registered trademark), 4G, or the like, which is a module used for communicating with another device. The display device 406 is a liquid crystal display, an organic light emitting diode (OLED) display, or the like and is used for display of a motion image, a static image, a text, a two-dimensional code, or the like. The input device 407 is a button, a touch panel, or the like and is used by the user to operate the user terminal 4. The display device 406 and the input device 407 may be integrally formed as a touch panel.

The visible light camera 408 is provided on a display face or the like of the display device 406, for example. The visible light camera 408 can capture a user's face, eye, or the like by using a visible light and acquire an image. For the visible light camera 408, a digital camera using a Complementary Metal Oxide Semiconductor (CMOS) image sensor, a Charge Coupled Device (CCD) image sensor, or the like may be used for suitable image capturing for subsequent image processing.

The infrared irradiation device 409 is a light emitting element that emits an infrared light, such as an infrared LED. For the infrared camera 410, a digital camera using a CMOS image sensor, a CCD image sensor, or the like having a light receiving element configured to have sensitivity to an infrared ray may be used. By irradiating the user's eye with an infrared ray from the infrared irradiation device 409 and capturing the infrared ray reflected by the iris by using the infrared camera 410, it is possible to capture an iris image used for iris authentication. Note that the wavelength of the infrared ray irradiated from the infrared irradiation device 409 may be within a near-infrared range around 800 nm, for example.

Note that the hardware configuration illustrated in FIG. 2 is an example, another device may be added, or some of the devices may not be provided. Further, some of the devices may be replaced with another device having the same function. Furthermore, some of the functions may be provided by another device via a network, or the function forming the present example embodiment may be distributed and implemented in a plurality of devices. For example, the flash memory 404 may be replaced with a hard disk drive (HDD) or may be replaced with cloud storage.

Each hardware configuration of the payment server 1 and the POS terminal 2 may include a computer having a CPU, a RAM, a ROM, an HDD, a communication I/F, an input device, an output device, or the like as with the user terminal 4.

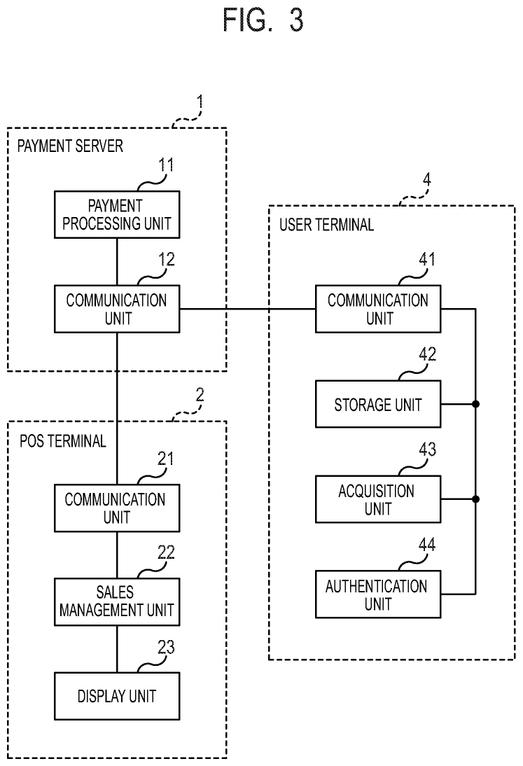

FIG. 3 is a function block diagram of the payment server 1, the POS terminal 2, and the user terminal 4 according to the first example embodiment. FIG. 3 depicts function blocks resulted from execution of a program by the CPU provided in each of the payment server 1, the POS terminal 2, and the user terminal 4.

The payment server 1 has a payment processing unit 11 and a communication unit 12. The payment processing unit 11 performs an electronic payment process of a transaction in response to a request from the user or a member store. The communication unit 12 communicates with the POS terminal 2 of a member store and the user terminal 4 of the user who purchases an item or the like. The CPU of the payment server 1 implements the function of the payment processing unit 11 by loading a program stored in the ROM or the like of the payment server 1 to the RAM and executing the program. The CPU of the payment server 1 implements the function of the communication unit 12 by controlling the communication I/F.

The POS terminal 2 has a communication unit 21, a sales management unit 22, and a display unit 23. The communication unit 21 communicates with the payment server 1. The sales management unit 22 supports management such as inventory management, sales management, or the like of a shop by performing a process when an item is sold and aggregating sales information. The display unit 23 is a display device such as a liquid crystal display, an OLED display, or the like provided to the POS terminal 2 and displays a text such as an item name, a price, a user name, a user identifier (ID), or the like, an image of a two-dimensional code corresponding to such text information, or the like. Here, an image of a two-dimensional code or the like includes information required for payment (payment-related information) such as an item name, a price, a user name, a user identifier (ID), shop information, or the like read by the POS terminal 2 from an item to be purchased by the user by using the barcode scanner or the like of the POS terminal 2. The CPU of the POS terminal 2 implements the function of the sales management unit 22 by loading a program stored in the ROM or the like of the POS terminal 2 to the RAM and executing the program. The CPU of the POS terminal 2 implements the function of the communication unit 21 by controlling the communication I/F.

The user terminal 4 has a communication unit 41, a storage unit 42, an acquisition unit 43, and an authentication unit 44. The communication unit 41 communicates with the payment server 1. The storage unit 42 stores an image captured by the visible light camera 408 or the infrared camera 410, feature data used for performing iris authentication, or the like. The acquisition unit 43 acquires an image by using the visible light camera 408, the infrared camera 410, or the like and stores the image in the storage unit 42. The authentication unit 44 performs iris authentication by calculating a feature of an iris image acquired by the infrared camera 410 and comparing the calculated feature with a feature of a pre-stored iris image used for comparison. The CPU 401 of the user terminal 4 implements the function of the authentication unit 44 by loading a program stored in the ROM 403, the flash memory 404, or the like of the user terminal 4 to the RAM 402 and executing the program. Further, the CPU 401 of the user terminal 4 implements the function of the acquisition unit 43 by controlling the visible light camera 408 and the infrared camera 410. Further, the CPU 401 of the user terminal 4 implements the function of the storage unit 42 by controlling the flash memory 404. Further, the CPU 401 of the user terminal 4 implements the function of the communication unit 41 by controlling the communication I/F 405. As described above, the user terminal 4 of the present example embodiment has the acquisition unit that acquires an image by using the visible light camera 408, the infrared camera 410, or the like and may be more generally referred to as an information acquisition system.

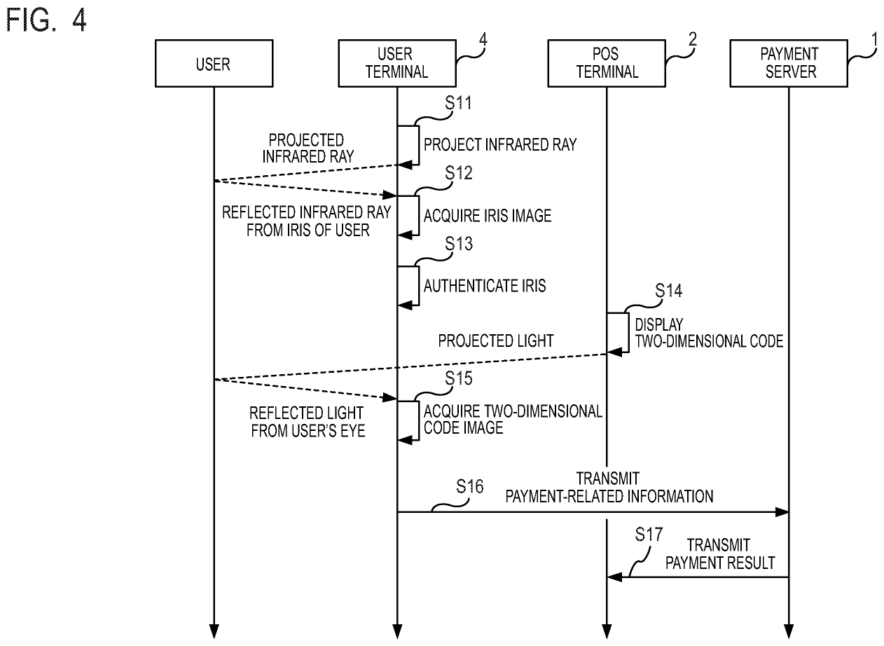

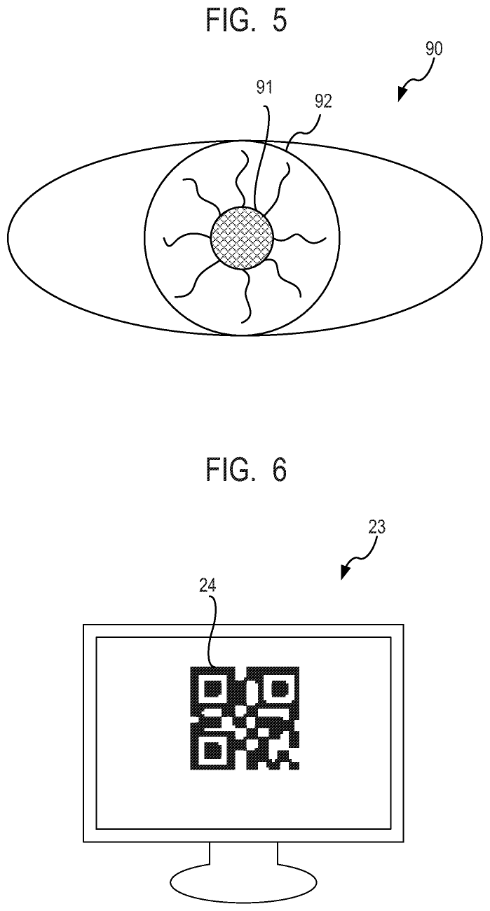

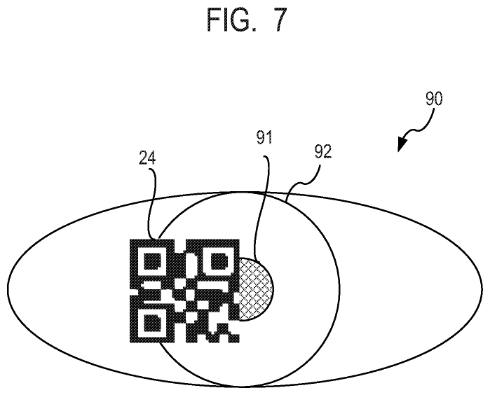

With reference to FIG. 4 to FIG. 7, a payment process of the present example embodiment will be described. FIG. 4 is a sequence diagram illustrating a payment process according to the present example embodiment. FIG. 5 is a diagram schematically illustrating an iris image according to the first example embodiment. FIG. 6 is a diagram schematically illustrating a display on which a two-dimensional code is displayed. FIG. 7 is a diagram schematically illustrating an image in which a two-dimensional code is reflected in an eye. FIG. 4 illustrates a process performed by the user terminal 4, the POS terminal 2, and the payment server 1. Each arrow indicated with a dashed line in FIG. 4 represents projection and capturing of a visible light or an infrared ray. With reference to FIG. 5, FIG. 6, and FIG. 7 if necessary, a payment process will be described in accordance with the time series in the sequence diagram of FIG. 4.

This payment process is a process in a situation where the user having the user terminal 4 visits a shop where the POS terminal 2 is installed and intends to purchase an item. A program for iris authentication and payment is installed in advance in the user terminal 4. At the time before step S11, the user operates the user terminal 4 in front of the POS terminal 2 and starts up the program in order to perform payment for purchase of an item. Note that the operation on the POS terminal 2 is performed by a salesclerk of the shop, for example.

In step S11 and step S12, the acquisition unit 43 of the user terminal 4 acquires an iris image of the user. More specifically, in step S11, the infrared irradiation device 409 of the user terminal 4 projects an infrared ray onto the periphery of the user's eye. In step S12, the infrared camera 410 of the user terminal 4 acquires an image (iris image) with an infrared ray reflected by the periphery of the user's eye including an iris. The iris image is stored in the storage unit 42 of the user terminal 4 and used for iris authentication of the user. Note that an iris image may be more generally referred to as a first image.

FIG. 5 illustrates a schematic diagram of an iris image captured with an infrared ray. As illustrated in FIG. 5, an image around an eye 90 is captured as an iris image with an infrared ray. The pattern of an iris 92 that adjusts the aperture of a pupil 91 is unique and permanent to an individual. Therefore, identity verification is possible by matching the pattern of the iris 92 acquired at authentication with an image of the iris 92 acquired in advance. Note that the reason why an infrared ray is used rather than a visible light for capturing an iris image is that a high contrast image is obtained regardless of the color of an iris and influence of reflection at a cornea can be reduced. For example, since it is difficult to achieve a high contrast with a visible light when the color of an iris is deep (black or the like), it is effective to use an infrared ray for capturing. On the other hand, when the color of an iris is light (blue or the like), a high contrast image may be obtained even with a visible light. An iris image may be acquired by using the visible light camera 408 when an iris image can be captured with a visible light without any problem, and it is not essential to use an infrared ray in capturing an iris. When the iris image is acquired by the visible light camera 408, the infrared irradiation device 409 and the infrared camera 410 can be omitted, and the device configuration can be simplified. Alternatively, when a camera having detection sensitivity also in the infrared range is employed as the visible light camera 408, the infrared camera 410 can be omitted, and the device configuration can be simplified. An example of such the visible light camera 408 may be a single-plate type CMOS image sensor having a pixel that detects an infrared light in addition to pixels of three colors that detect red, green, and blue visible lights.

In step S13, the authentication unit 44 of the user terminal 4 performs authentication by matching the iris image acquired by the process of step S12 with an iris image of the user acquired in advance. When the authentication fails, a payment process is not performed, and instead an operation such as requesting re-authentication, notifying the user that the authentication failed and suspending the process, or the like may be performed. When the authentication is successful, the process proceeds to the next process.

In step S14, the POS terminal 2 displays an image of a two-dimensional code of a QR code (registered trademark) or the like on the display unit 23. FIG. 6 illustrates a display example of a two-dimensional code 24 on the display unit 23. The two-dimensional code 24 includes information required for payment (payment-related information) such as an item name, a price, a user name, a user identifier (ID), shop information, or the like. Such payment-related information included in the two-dimensional code 24 is information read by the POS terminal 2 from an item to be purchased by the user by using a barcode scanner or the like of the POS terminal 2. That is, the information included in the two-dimensional code 24 is information used for a different purpose from biometrics authentication of the user. When the user watches the display unit 23 or directs its face thereto, a light projected from the display unit 23 is reflected by the user's eye (for example, the cornea). Thereby, an image of the two-dimensional code 24 is reflected in the user's eye. Note that an image reflected in the user's eye may be more generally referred to as a second image.

In step S15, the visible light camera 408 of the user terminal 4 acquires an image including the two-dimensional code 24 reflected in the user's eye. The image including the two-dimensional code 24 is stored in the storage unit 42 of the user terminal 4. As illustrated in FIG. 7, the two-dimensional code 24 is reflected in the user's eye. The user terminal 4 can acquire payment-related information from the two-dimensional code 24. Note that, if possible, the image including the two-dimensional code 24 may be captured by the infrared camera 410.

In step S16, the communication unit 41 of the user terminal 4 transmits the payment-related information included in the two-dimensional code 24 acquired in step S15 to the payment server 1. The communication unit 12 of the payment server 1 receives the payment-related information, and the payment processing unit 11 performs payment for the purchase of the item by the user based on the payment-related information. Then, in step S17, the communication unit 12 of the payment server 1 transmits a payment result to the POS terminal 2. The communication unit 21 of the POS terminal 2 receives the payment result, the sales management unit 22 of the POS terminal 2 completes the sales process on the item, and the display unit 23 displays the completion of the sales process. The salesclerk operating the POS terminal 2 passes the item to the user upon the completion of the process.

In the present example embodiment, payment-related information is included in an image, and it is possible to transfer the payment-related information to from the POS terminal 2 to the user terminal 4 by using reflection at the user's eye. In such a way, the user terminal 4 that performs biometrics authentication can acquire the payment-related information, which is not held in advance, without directly communication with the POS terminal 2, and the communication configuration can be simplified. Further, iris authentication has been performed by the user terminal 4, and it is therefore not necessary for the POS terminal 2 side to hold data used for iris authentication of the user. Thus, the amount of data to be stored in the POS terminal 2 can be reduced.

As described above, the user terminal 4 of the present example embodiment can acquire information used for a different purpose from biometrics authentication in addition to acquisition of information used for biometrics authentication.

Further, in the present example embodiment, when impersonation is attempted at iris authentication by a scheme of using a photograph of a user's eye or the like, an image of a two-dimensional code reflected by an eye cannot be acquired, payment is thus disabled, and therefore an advantage of suppressing impersonation is also obtained.

Note that the payment-related information may not be a two-dimensional code but may be displayed in another form of a code such as a one-dimensional code or may be displayed in a text or the like. With the display of a two-dimensional code, however, error detection and correction can be performed, and an advantage of an increased probability that information can be correctly transferred even when distortion of an image occurs due to reflection in an eye is obtained. Further, when the payment server 1 or the user terminal 4 can acquire information required for payment from the shape of an item, the user terminal 4 may acquire an image of the item reflected in the user's eye that is watching the item.

Further, information included in a two-dimensional code may include additional information used for campaign, such as a point given as a privilege to the user, and not limited to information required for payment. Further, a two-dimensional code may further include information such as an identification information (ID) of a device such as the POS terminal 2 involved in a transaction associated with payment, sales time (time information), location information on the POS terminal 2 or the like, or the like. With reference to the above information, the time, the location, or the like of authentication is clarified, and an advantage of preventing wrong authentication is obtained. Further, a two-dimensional code may include a onetime password. With inclusion of a onetime password, an advantage of preventing impersonation using a photograph, a motion image, or the like is obtained.

In step S16, the transmission of payment-related information may be to transmit the image of a two-dimensional code, which may be configured such that the two-dimensional code is analyzed by the user terminal 4 and information included in the two-dimensional code is transmitted but the image is not transmitted.

The configuration of the user terminal 4 can take various forms. For example, a wearable terminal of a glasses type or the like may be used, and in such a case, it is possible to more easily capture an image of an eye.

The functions of the storage unit 42, the acquisition unit 43, and the authentication unit 44 may be provided on the POS terminal 2 side. In such a modified example, display of a two-dimensional code is performed on the user terminal 4 side, and the two-dimensional code reflected by the user's eye is acquired by the POS terminal 2. In such a case, the two-dimensional code may include information on an item that the user intends to purchase, authentication information on the user, or the like, for example, and such information can be transferred from the user terminal 4 to the POS terminal 2 without direct communication between the POS terminal 2 and the user terminal 4.

Further, all the functions of the user terminal 4 may be included in the POS terminal 2, and thereby the user terminal 4 may be omitted from the system configuration of the present example embodiment. In such a configuration, the functions of the storage unit 42, the acquisition unit 43, and the authentication unit 44 are provided on the POS terminal 2 side, and moreover, display of a two-dimensional code and acquisition of an image are performed by the POS terminal 2. In such a modified example, iris authentication is performed by an iris image acquired by the camera of the POS terminal 2. In addition, the two-dimensional code is displayed on the display unit 23 of the POS terminal 2, and the two-dimensional code reflected by the user's eye is acquired by the camera of the POS terminal 2. Such iris authentication and acquisition of a two-dimensional code may be performed at the same time. The feature of the user's iris used for iris authentication may be acquired by storing the feature in the POS terminal 2 in advance or accessing a data server having the feature of the user's iris at iris authentication and downloading the feature. According to this modified example, iris authentication and payment can be performed in parallel in a series of processes or performed at the same time. Note that the camera that acquires a two-dimensional code may be a visible light camera or may be an infrared camera.

Further, the function of the display unit 23 of the POS terminal 2 may be included in the user terminal 4, and thereby display of a two-dimensional code may be performed by the user terminal 4. In such a modified example, iris authentication is performed by using an iris image acquired by the infrared camera 410 of the user terminal 4. In addition, the two-dimensional code is displayed on the display device 406 of the user terminal 4, and the two-dimensional code reflected by the user's eye is acquired by the visible light camera 408 of the user terminal 4. The two-dimensional code displayed on the display device 406 of the user terminal 4 may be acquired by the user terminal 4 communicating with the POS terminal 2 or the payment server 1 at payment to perform this process. Such iris authentication and acquisition of a two-dimensional code may be performed at the same time. Also in this modified example, iris authentication and payment can be performed in parallel in a series of processes or performed at the same time. Note that the camera that acquires a two-dimensional code may be a visible light camera or may be an infrared camera.

Note that, in the configuration of displaying a two-dimensional code on the user terminal 4, information of the two-dimensional code displayed on the display device 406 of the user terminal may be acquired by the user terminal 4 communicating with the POS terminal 2 or the payment server 1 before payment. However, this information of the two-dimensional code may be acquired by using the visible light camera 408 of the user terminal 4 to capture an item displayed in the shop or a barcode, a two-dimensional code, or the like attached thereto, or information associated with an item displayed in the shop may be acquired by an application installed in the user terminal 4. As an example of a specific situation, the user picks up an item to be purchased and causes the user terminal 4 to read a barcode including item information or the like, and thereby a two-dimensional code including payment-related information on the item to be purchased is displayed on the user terminal 4. The camera of the POS terminal 2 or the user terminal 4 performs iris authentication and further acquires the two-dimensional code reflected in the user's eye. The POS terminal 2 or the user terminal 4 can perform iris authentication and acquire payment-related information included in the two-dimensional code in such a way. Note that the information of the two-dimensional code to be displayed on the user terminal 4 may be acquired from an entity other than an item displayed in the shop. For example, payment-related information may be acquired via a network by so-called internet shopping, and a two-dimensional code related thereto may be displayed on the user terminal 4. This configuration may be mainly applied to a case of shop payment in which acquisition of payment-related information is performed in internet shopping and payment is performed by the POS terminal 2 of the shop such as a convenience store.

The system configuration illustrated above is an example, and it is possible to appropriately set which device of the user terminal 4 or the POS terminal 2 performs each process, and a part of the process may be performed by a separate device other than the above terminals.

The iris image acquisition and authentication of step S11 to step S13 and acquisition of a two-dimensional code image of step S14 and step S15 may be performed in the opposite order or may be performed in parallel.

Further, after the iris image acquisition and authentication of step S11 to step S13 are performed, acquisition of a two-dimensional code image of step S14 and step S15 may be repeated for multiple times. Alternatively, the process of step S14 to step S17 may be repeated for multiple times. In such a way, by repeating only the acquisition of a two-dimensional code image for multiple times, the user terminal 4 of the present example embodiment can acquire information used for a different purpose from biometrics authentication for multiple times after performing acquisition of information used for biometrics information once. This modified example is effective for application to a use in which multiple times of information acquisition are performed after one time of biometrics authentication. For example, in a situation where a user completes check once and then suddenly wants to additionally purchase another item in a convenience store, since it is no longer necessary to again perform biometrics authentication, this improves convenience.

### [Second Example Embodiment]

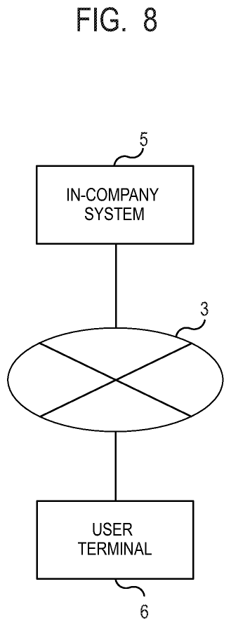

As a second example embodiment of the present invention, an example of a telework system that performs user management using iris authentication will be described. FIG. 8 is a schematic diagram illustrating a general configuration of the telework system according to the second example embodiment. The telework system includes an in-company system 5 and a user terminal 6. The user terminal 6 is connected to the in-company system 5 via a network 3. This telework system provides a teleworking environment in a form of telecommuting, freelance work, or the like to the user terminal 6. A user operating the user terminal 6 is able to access and work in the in-company system 5 from a remote location.

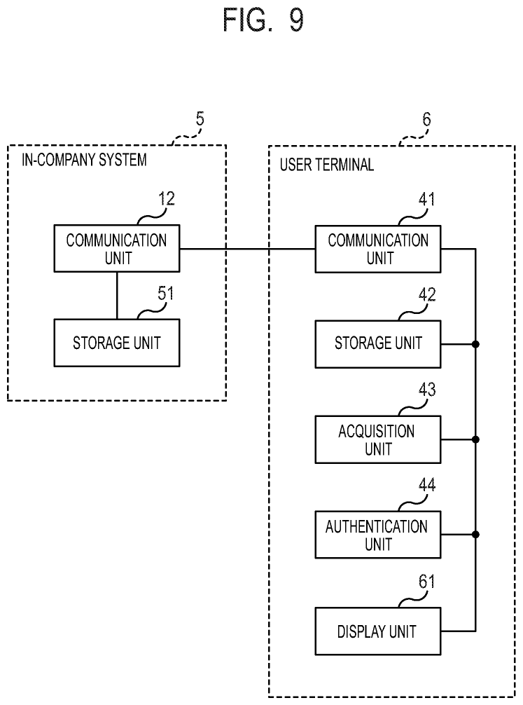

FIG. 9 is a function block diagram of the in-company system 5 and the user terminal 6 according to the present example embodiment. FIG. 9 depicts function blocks resulted from execution of programs by the CPU provided in each of the in-company system 5 and the user terminal 6. Note that, since the same configuration as that of the first example embodiment may be applied for the hardware configuration, the description thereof will be omitted.

The in-company system 5 has the communication unit 12 and a storage unit 51. The communication unit 12 communicates with the user terminal 6 that accesses the in-company system 5. The storage unit 51 stores data required for performing an operation. Further, the storage unit 51 further stores data used for managing teleworking transmitted from the user terminal 6. The CPU of the in-company system 5 implements the functions of the communication unit 12 and the storage unit 51 by controlling a communication I/F and a nonvolatile storage medium.

The user terminal 6 of the present example embodiment further has a display unit 61 in addition to the configuration of the user terminal 4 of the first example embodiment. The display unit 61 is a display device such as a liquid crystal display, an OLED display, or the like and displays data used for operation, a work screen, or the like acquired from the in-company system 5. The user terminal 6 of the present example embodiment has the acquisition unit 43 that acquires an image and may be more generally referred to as an information acquisition system.

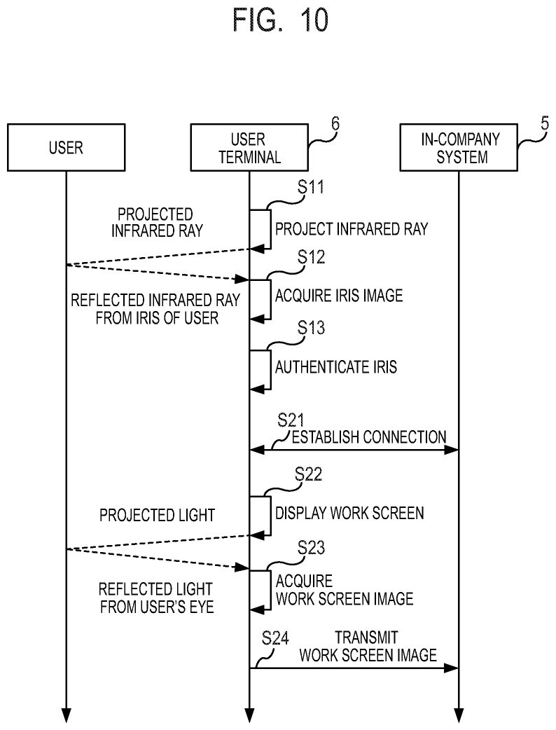

FIG. 10 is a sequence diagram illustrating a user management process according to the present example embodiment. The user management process will be described in accordance with the time series in the sequence diagram of FIG. 10. This user management process is performed when the user operating the user terminal 6 attempts to access the in-company system 5 for teleworking and during work of the teleworking. A program for iris authentication and network connection is installed in advance in the user terminal 6. At the time before step S11, the user operates the user terminal 6 and starts up the program in order to start teleworking.

In step S11 and step S12, the acquisition unit 43 of the user terminal 6 acquires an iris image of the user who is an authentication target. In step S13, the authentication unit 44 of the user terminal 6 performs authentication by matching the iris image acquired by the process of step S12 with an iris image of the user acquired in advance. Note that an iris image may be more generally referred to as a first image. Since these processes are the same as those in the first example embodiment, detailed description thereof will be omitted.

If the authentication is successful, connection between the user terminal 6 and the in-company system 5 is established, and teleworking is ready for start (step S21). During work of the subsequent teleworking, acquisition of a work screen of step S22 to step S24 is performed for monitoring a working state. This acquisition of a work screen is performed at regular intervals (for example, intervals of 15 minutes, intervals of 30 minutes, or the like), for example.

In step S22, the display unit 61 of the user terminal 6 displays a work screen. This display of the work screen may be a work screen of the contents of teleworking or may further include display of another information. A light reflected from the display unit 61 is reflected by the user's eye (for example, the cornea). Thereby, an image of the work screen is reflected in the user's eye. Note that an image reflected in a user's eye may be more generally referred to as a second image.

In step S23, the acquisition unit 43 of the user terminal 6 acquires an image including the work screen reflected in the user's eye (a work screen image). The work screen image is stored in the storage unit 42 of the user terminal 6.

In step S24, the communication unit 41 of the user terminal 6 transmits the work screen image acquired in step S23 to the in-company system 5. The communication unit 12 of the in-company system 5 receives the work screen image. The work screen image is stored in the storage unit 51.

A work screen image is used for management so that teleworking is properly performed. For example, a manager of the in-company system 5 is able to check the content of the work screen image and monitor whether or not the user is working hard on teleworking. Specifically, when no work screen is reflected suitably in the user's eye, the user is not facing the work screen, and thus it can be determined that the user is less likely to be working hard on the teleworking. Alternatively, by checking whether or not the content of the work screen image is an appropriate content that relates to the operation, it is also possible to determine whether or not the user is working hard on the teleworking. When the user is not working hard on the teleworking, it is possible to display an alert message on the display unit 61 of the user terminal 6 to urge the user to work hard, for example. Note that checking of a work screen image may be automatically performed by using an image recognition technology.

In the present example embodiment, an image of a work screen on the user terminal 6 can be acquired by using reflection by the user's eye. The information included in the work screen image acquired in such a way can be used for management so as to cause teleworking to be performed appropriately.

As described above, also in the user terminal 6 of the present example embodiment, it is possible to acquire information used for a different purpose from biometrics authentication in addition to acquisition of information used for biometrics authentication.

Further, also in the present example embodiment, when impersonation is attempted at iris authentication with a scheme of using a photograph of a user's eye or the like, a work screen image reflected by an eye cannot be acquired, and thus the advantage of suppressing impersonation is also obtained. Note that, when impersonation is detected, the in-company system 5 can shut off the connection between the user terminal 6 and the in-company system 5.

In step S22, for example, a text, a one-dimensional code, a two-dimensional code, or the like may be added to the work screen displayed on the display unit 61 in the same manner as in the case of the first example embodiment. When a coded image such as a one-dimensional code, a two-dimensional code, or the like is displayed, this may include information indicating the ID of the user terminal 6, time, location information, a onetime password, and the content of work, information changing with time, or the like. Since such information is not reflected in a case of impersonation using a sheet or a display, an advantage of preventing impersonated unauthorized connection is obtained. Further, for example, by matching the time acquired from the two-dimensional code or the like with the transmission time of an image, it is possible to detect unauthorized alteration. Further, when a coded image includes information indicating the content of work or information changing with time, the manager of the in-company system 5 can monitor whether or not the code including the information indicating the content of work or the information changing with time is reflected in the user's eye and thereby monitor whether or not the user is appropriately performing the operation. Alternatively, the manager of the in-company system 5 may match the above information included in the code reflected in the user's eye with the actual content of work or time of work and thereby monitor whether or not the user is appropriately performing the operation.

A screenshot image displayed on the display unit 61 of the user terminal 6 may be further acquired in step S23, and the screenshot image may be further transmitted from the user terminal 6 to the in-company system 5 in step S24. In the in-company system 5, by matching the screenshot image with a work screen image reflected in the eye, it is possible to more reliably prevent impersonation.

The iris authentication of step S11 to step S13 may be performed after the establishment of connection of step S21, or the acquisition of a work screen image of step S22 to step S24 may be performed before the establishment of connection of step S21 or at the time of the establishment of the connection.

In the process of the present example embodiment, a step of transmitting an iris image or a feature from the user terminal 6 to the in-company system 5 may be further included, and in such a case, the function of the authentication unit 44 may be provided on the in-company system 5 side.

The configuration of the user terminal 6 can take various forms. For example, a wearable terminal of a glasses type or the like may be used, and in such a case, it is possible to more easily capture an image of an eye.

The function of the user terminal 6 may be distributed and provided in a plurality of devices. The system configuration of such a case may employ various configurations in accordance with a combination of functions provided in each device as illustrated as a plurality of examples in the first example embodiment. Therefore, the system configuration of the present example embodiment is not limited to that illustrated in FIG. 10.

### [Third Example Embodiment]

As a third example embodiment of the present invention, an example of an entry/exit management system using iris authentication will be described. The entry/exit management system of the present example embodiment relates to authentication at entry to or exit from a facility or the like and may be applied to a situation of entry to or exit from an event site such as a concert, entry to or exit from a theme park, entry to or exit from a factory or an office, entry into or departure from a country at an airport, a seaport, or a national border, or the like, for example. While a situation of iris authentication at entry to an event site will be described below as an example, the example embodiment is applicable similarly to another purpose as long as it is a situation such as entry or exit where biometrics authentication is required.

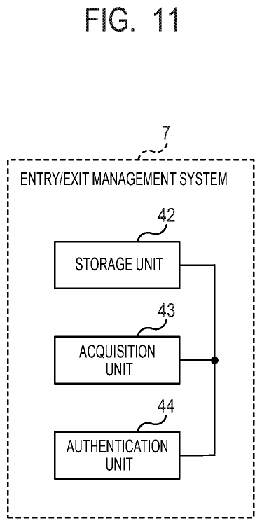

FIG. 11 is a function block diagram of an entry/exit management system 7 according to the present example embodiment. FIG. 11 depicts function blocks resulted from execution of programs by the CPU provided in the entry/exit management system 7. The entry/exit management system 7 of the present example embodiment has the storage unit 42, the acquisition unit 43, and the authentication unit 44 as with the user terminal 4 of the first example embodiment. Since the same configuration as that of the first example embodiment may be applied for the details of each component and the hardware configuration, the description thereof will be omitted. The entry/exit management system 7 of the present example embodiment has the acquisition unit 43 that acquires an image and thus may be more generally referred to as an information acquisition system.

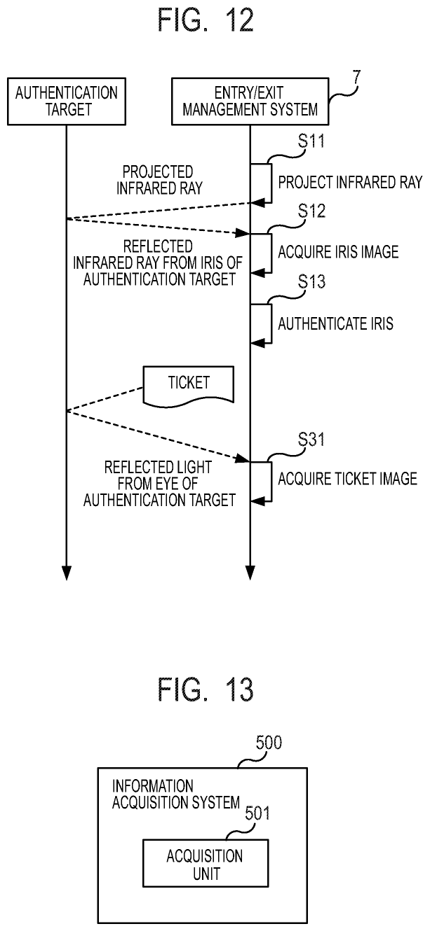

FIG. 12 is a sequence diagram illustrating an entry/exit management process according to the present example embodiment. The entry/exit management process will be described in accordance with the time series of the sequence diagram of FIG. 12. This entry/exit management process is performed on an authentication target who holds a ticket and intends to enter an event site. A program for iris authentication for the entry/exit management process and acquisition of an image of a ticket is installed in advance in the entry/exit management system 7. At the time before step S11, the manager of the entry/exit management system 7 operates the entry/exit management system 7 and starts up the program.

In step S11 and step S12, the acquisition unit 43 of the entry/exit management system 7 acquires an iris image of the user who is an authentication target. In step S13, the authentication unit 44 of the entry/exit management system 7 performs authentication by matching the iris image acquired by the process of step S12 with an iris image of the authentication target acquired in advance. Note that an iris image may be more generally referred to as a first image. Since these processes are the same as those in the first example embodiment, detailed description thereof will be omitted.

The authentication target holds a ticket used for participating in an event. In the ticket, information such as an event name, a date and time of the event, a ticket ID, a seat number, or the like is written. Due to diffused reflection of lighting or natural light on the ticket, a light projected from the ticket is reflected by an eye (for example, a cornea) of the authentication target. Thereby, an image of the ticket is reflected in the eye of the authentication target. Note that an image reflected in an eye of an authentication target may be more generally referred to as a second image.

In step S31, the acquisition unit 43 of the entry/exit management system 7 acquires the image of the ticket reflected in the eye of the authentication target (ticket image). The ticket image is stored in the storage unit 42 of the entry/exit management system 7.

The entry/exit management system 7 can use information acquired from the ticket image by using Optical Character Recognition (OCR) or the like for various purposes. For example, when acquiring an event name or a date and time of the event, it is possible to use the information for the purpose of seeing if the authentication target is not making a mistake on the event to participate in. When acquiring a ticket ID, it is possible to use the information for the purpose of checking whether or not there is a matching with an authentication target associated with an iris image and thereby detecting whether or not the ticket is fraudulently resold, the ticket is forged, or the like. When acquiring a seat number, it is possible to use the information for the purpose of guiding a visitor to a correct seat.

In the present example embodiment, a ticket image can be acquired by using reflection by an eye of an authentication target. The information included in the ticket image acquired in such a way can be used for various purposes different from biometrics authentication.

As described above, also in the entry/exit management system 7 of the present example embodiment, it is possible to acquire information used for a different purpose from biometrics authentication in addition to acquisition of information used for biometrics authentication.

Further, in the present example embodiment, when impersonation is attempted at iris authentication with a scheme of using a photograph of an eye of an authentication target or the like, a ticket image reflected by an eye cannot be acquired, and thus an advantage of suppressing impersonation is also obtained. Note that, when impersonation is detected, a countermeasure to prohibit entry or exit may be taken.

A coded image such as a one-dimensional code, a two-dimensional code, or the like may be added to a ticket in addition to text information. With purchaser information, a password, or the like being included in the above, an advantage of preventing fraudulent resale, forgery, or the like is obtained.

The entry/exit management system 7 may be formed of a plurality of devices, for example, may be formed of a terminal that acquires an iris image and a ticket image and a server that performs a process of authentication or the like.

The iris authentication of step S11 to step S13 may be performed in parallel to the acquisition of a ticket image of step S31 or may be performed after the acquisition of a ticket image of step S31.

An image reflected in an eye of an authentication target is not limited to a ticket. For example, in a situation of entry to or exit from a factory or an office, the image may be an admission card or an identification card. In a situation of entry into or departure form a country or the like at an airport, a seaport, or a national border, the image may be a boarding pass, a passport, an immigration document, or the like. Further, an image reflected in an eye of an authentication target may not be a document, a card, or the like. For example, when the authentication target has a onetime password generator, the image reflected in an eye of an authentication target may include a password displayed on the onetime password generator.

The function of the entry/exit management system 7 may be distributed and provided in a plurality of devices. The system configuration of such a case may employ various configurations in accordance with a combination of functions provided in each device as illustrated as a plurality of examples in the first example embodiment. Therefore, the system configuration of the present example embodiment is not limited to that illustrated in FIG. 12.

### [Fourth Example Embodiment]

The device described in the above example embodiments can also be configured as below. FIG. 13 is a function block diagram of an information acquisition system 500 according to a fourth example embodiment. The information acquisition system 500 has an acquisition unit 501. The acquisition unit 501 acquires a first image that includes biometrics information and is used for biometrics authentication of an authentication target and a second image that is based on a light projected onto and reflected by a body of the authentication target and includes information used for a different purpose from the biometrics authentication of the authentication target.

According to the present example embodiment, an information acquisition system that can acquire information used for a different purpose from biometrics authentication in addition to acquisition of information used for biometrics authentication can be provided.

### [Modified Example Embodiments]

The present invention is not limited to the example embodiments described above and can be appropriately changed within the scope not departing from the scope of the present invention.

The biometrics authentication that may be performed in each of the example embodiments described above is not limited to iris authentication and may be face authentication, for example. Further, acquisition of an image including information used for a different purpose from biometrics authentication is not limited to that based on a reflected light from an eye of an authentication target and may be any acquisition as long as it is based on a light reflected by the body thereof. For example, a reflected light from a face of an authentication target may be used. In such a case, to ensure the intensity of the reflected light, a laser light may be used as a projection light to the authentication target.

In each of the example embodiments described above, acquisition of an image used for iris authentication and acquisition of an image reflected in an eye may be performed on one of the eyes of an authentication target or may be performed on both of the eyes. When only the image of one of the eyes is acquired, there are advantages of improved processing speed and reduced storage amount, and when images of both of the eyes are acquired, there is an advantage of improved authentication accuracy.

In each of the example embodiments described above, since the surface of an eye is a curved surface, an image reflected in the eye may be distorted. Accordingly, image processing to correct the distortion of the curved surface of the eye after acquiring an image reflected in the eye may be added. Further, image processing to correct a contrast caused solely by the eye, such as a difference in the color of a pupil, an iris, and a sclera, may be added.

The user terminal 4 in the first example embodiment is a terminal that may be used in a situation of payment. The user terminal 4 may be typically a smartphone or a tablet terminal but is not limited thereto. Further, the user terminal 6 in the second example embodiment is a terminal that may be used for teleworking. The user terminal 6 may be typically a notebook PC but is not limited thereto.

Further, the scope of each of the example embodiments includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the program itself. Further, one or more components included in the example embodiments described above may be a circuit such as an Application Specific Integrated Circuit (ASIC), a Field Programmable Gate Array (FPGA), or the like configured to implement the function of each component.

As the storage medium, for example, a floppy (registered trademark) disk, a hard disk, an optical disk, a magneto-optical disk, a compact disk (CD)-ROM, a magnetic tape, a nonvolatile memory card, or a ROM can be used. Further, the scope of each of the example embodiments includes an example that operates on an operation system (OS) to perform a process in cooperation with another software or a function of an add-in board without being limited to an example that performs a process by an individual program stored in the storage medium.

The service implemented by the function of each of the example embodiments described above may be provided to the user in a form of Software as a Service (SaaS).

Note that each of the example embodiments described above merely illustrates an example of embodiments in implementing the present invention, and the technical scope of the present invention should not be construed in a limiting sense by these example embodiments. That is, the present invention can be implemented in various forms without departing from the technical concept or the primary features.

The whole or part of the example embodiments disclosed above can be described as, but not limited to, the following supplementary notes.

### (Supplementary Note 1)

An information acquisition system comprising an acquisition unit that acquires a first image used for biometrics authentication of an authentication target and a second image that is based on a light projected onto and reflected by a body of the authentication target and includes information used for a different purpose from the biometrics authentication of the authentication target.

### (Supplementary Note 2)

The information acquisition system according to supplementary note 1, wherein the first image includes an image of an iris of the authentication target.

### (Supplementary Note 3)

The information acquisition system according to supplementary note 1 or 2, wherein the second image includes an image reflected in an eye of the authentication target.

### (Supplementary Note 4)

The information acquisition system according to supplementary note 1, wherein the first image includes an image of a face of the authentication target, and the second image includes an image reflected in the face of the authentication target.

### (Supplementary Note 5)

The information acquisition system according to any one of supplementary notes 1 to 4, wherein the second image includes information which is not held in advance by a device that performs biometrics authentication of the authentication target.

### (Supplementary Note 6)

The information acquisition system according to any one of supplementary notes 1 to 5, wherein the second image includes a two-dimensional code.

### (Supplementary Note 7)

The information acquisition system according to any one of supplementary notes 1 to 6, wherein the second image includes payment-related information related to payment performed after the biometrics authentication.

### (Supplementary Note 8)

The information acquisition system according to supplementary note 7, wherein the payment-related information includes at least one of an item name and a price.

### (Supplementary Note 9)

The information acquisition system according to supplementary note 7 or 8, wherein the payment-related information includes at least one of identification information on a device related to a transaction associated with the payment, time information related to the transaction, and location information related to the transaction.

### (Supplementary Note 10)

The information acquisition system according to any one of supplementary notes 1 to 7, wherein the second image is an image indicating a shape of an item.

### (Supplementary Note 11)

The information acquisition system according to any one of supplementary notes 1 to 6, wherein the second image includes information displayed on a display unit of a terminal operated by the authentication target.

### (Supplementary Note 12)

The information acquisition system according to supplementary note 11, wherein the second image includes information that changes in accordance with at least one of a time and a content of work performed by the authentication target using the terminal.

### (Supplementary Note 13)

The information acquisition system according to any one of supplementary notes 1 to 6, wherein the second image includes information written in a ticket possessed by the authentication target.

### (Supplementary Note 14)

The information acquisition system according to any one of supplementary notes 1 to 13, wherein the acquisition unit acquires the second image for multiple times after acquiring the first image once.

### (Supplementary Note 15)

An information acquisition method comprising:
acquiring a first image used for biometrics authentication of an authentication target; and
acquiring a second image that is based on a light projected onto and reflected by a body of the authentication target and includes information used for a different purpose from the biometrics authentication of the authentication target.

### (Supplementary Note 16)

A storage medium storing a program that causes a computer to perform:
acquiring a first image used for biometrics authentication of an authentication target; and
acquiring a second image that is based on a light projected onto and reflected by a body of the authentication target and includes information used for a different purpose from the biometrics authentication of the authentication target.

This application is based upon and claims the benefit of priority from Japanese Patent Application No. 2017-155190, filed on Aug. 10, 2017, the disclosure of which is incorporated herein in its entirety by reference.

### REFERENCE SIGNS LIST

1 payment server
2 POS terminal
3, 3a, 3b network
4 user terminal
5 in-company system
6 user terminal
7 entry/exit management system
11 payment processing unit
12, 21, 41 communication unit
22 sales management unit
23 display unit
24 two-dimensional code
42, 51 storage unit
43, 501 acquisition unit
44 authentication unit
90 eye
91 pupil
92 iris
61 display unit
401 CPU
402 RAM
403 ROM
404 flash memory
405 communication I/F
406 display device
407 input device
408 visible light camera
409 infrared irradiation device
410 infrared camera
411 bus
500 information acquisition system

1. A glasses device comprising:
a display configured to display an image;
a camera configured to capture an eye of a user wearing the glasses device;
at least one memory storing instructions; and
at least one processor executing the instructions to:
capture an image of the eye of the user for authentication of the user by the camera;
display, on the display, an image for the user received from an external device via a network in response to authentication of the user being successful based on the image of the eye of the user for authentication of the user;
capture an image by the camera after displaying, on the display, the image for the user received from the external device via the network; and
display an alert on the display based on the image for the user received from the external device and the image captured by the camera.

2. The glasses device according to claim 1, wherein the instruction to display the alert is executed in response to whether the image captured by the camera includes at least a part of the image for the user received from the external device.

3. The glasses device according to claim 2, wherein the image captured by the camera shows the eye of the user.

4. The glasses device according to claim 1, wherein the at least one processor further executes the instructions to transmit an image displayed on the display to the external device in response to acquiring the image displayed on the display.

5. The glasses device according to claim 4, wherein the image displayed on the display includes a screenshot image displayed on the display.

6. A method of controlling a glasses device including a display configured to display an image, and a camera configured to capture an eye of a user wearing the glasses device, the method comprising:
capturing an image of the eye of the user for authentication of the user by the camera;
displaying, on the display, an image for the user received from an external device via a network in response to authentication of the user being successful based on the image of the eye of the user for authentication of the user;
capturing an image by the camera after displaying, on the display, the image for the user received from the external device via the network; and
displaying an alert on the display based on the image for the user received from the external device and the image captured by the camera.

7. The method according to claim 6, wherein the displaying the alert is executed in response to whether the image captured by the camera includes at least a part of the image for the user received from the external device.

8. The method according to claim 7, wherein the image captured by the camera shows the eye of the user.

9. The method according to claim 6 further comprising transmitting an image displayed on the display to the external device in response to acquiring the image displayed on the display.

10. The method according to claim 9, wherein the image displayed on the display includes a screenshot image displayed on the display.

11. A non-transitory storage medium storing a program that, when executed by a computer that controls a glasses device including a display configured to display an image, and a camera configured to capture an eye of a user wearing the glasses device, causes the computer to perform:
capturing an image of the eye of the user for authentication of the user by the camera;
displaying, on the display, an image for the user received from an external device via a network in response to authentication of the user being successful based on the image of the eye of the user for authentication of the user;
capturing an image by the camera after displaying, on the display, the image for the user received from the external device via the network; and
displaying an alert on the display based on the image for the user received from the external device and the image captured by the camera.

12. The non-transitory storage medium according to claim 11, wherein the displaying the alert is executed in response to whether the image captured by the camera includes at least a part of the image for the user received from the external device.

13. The non-transitory storage medium according to claim 12, wherein the image captured by the camera shows the eye of the user.

14. The non-transitory storage medium according to claim 11, the program further causes the computer to perform transmitting an image displayed on the display to the external device in response to acquiring the image displayed on the display.

15. The non-transitory storage medium according to claim 14, wherein the image displayed on the display includes a screenshot image displayed on the display.
